SEQUENCE LISTING (84 sequences)

SEQ ID NO: 1 - DNA, 21 bp, Artificial Sequence
  Description of Combined DNA/RNA Molecule Synthetic oligonucleotide
cnacacaaancagcgannntt

SEQ ID NO: 2 - DNA, 21 bp, Artificial Sequence
  Description of Combined DNA/RNA Molecule Synthetic oligonucleotide
aaancgcngannngngnagtt

SEQ ID NO: 3 - DNA, 21 bp, Artificial Sequence
  Description of Combined DNA/RNA Molecule Synthetic oligonucleotide
cnacacaaancagcgannntt

SEQ ID NO: 4 - DNA, 21 bp, Artificial Sequence
  Description of Combined DNA/RNA Molecule Synthetic oligonucleotide
aaaucgcugauuuguguagtt

SEQ ID NO: 5 - DNA, 21 bp, Artificial Sequence
  Description of Combined DNA/RNA Molecule Synthetic oligonucleotide
cuacacaaaucagcgauuutt

SEQ ID NO: 6 - DNA, 21 bp, Artificial Sequence
  Description of Combined DNA/RNA Molecule Synthetic oligonucleotide
aaancgcngannngngnagtt

SEQ ID NO: 7 - DNA, 21 bp, Artificial Sequence
  Description of Combined DNA/RNA Molecule Synthetic oligonucleotide
cuacacaaaucagcgauuutt

SEQ ID NO: 8 - DNA, 21 bp, Artificial Sequence
  Description of Combined DNA/RNA Molecule Synthetic oligonucleotide
aaaucgcugauuuguguagtt

SEQ ID NO: 9 - DNA, 20 bp, Artificial Sequence
  Description of Combined DNA/RNA Molecule Synthetic oligonucleotide
gcannnggcanaagaaantt

SEQ ID NO: 10 - DNA, 20 bp, Artificial Sequence
  Description of Combined DNA/RNA Molecule Synthetic oligonucleotide
annnncnnangccaaanctt

SEQ ID NO: 11 - DNA, 21 bp, Artificial Sequence
  Description of Combined DNA/RNA Molecule Synthetic oligonucleotide
ccngcngcnangccncanctt

SEQ ID NO: 12 - DNA, 21 bp, Artificial Sequence
  Description of Combined DNA/RNA Molecule Synthetic oligonucleotide
gangaggcanagcagcaggtt

SEQ ID NO: 13 - DNA, 21 bp, Artificial Sequence
  Description of Combined DNA/RNA Molecule Synthetic oligonucleotide
nnnggaaaggaccagcaaatt

SEQ ID NO: 14 - DNA, 21 bp, Artificial Sequence
  Description of Combined DNA/RNA Molecule Synthetic oligonucleotide
nnngcnggnccnnnccaaatt

SEQ ID NO: 15 - DNA, 21 bp, Artificial Sequence
  Description of Combined DNA/RNA Molecule Synthetic oligonucleotide
cacccngacaagcngccagtt

SEQ ID NO: 16 - DNA, 21 bp, Artificial Sequence
  Description of Combined DNA/RNA Molecule Synthetic oligonucleotide
cnggcagcnngncagggngtt

SEQ ID NO: 17 - DNA, 21 bp, Artificial Sequence
  Description of Combined DNA/RNA Molecule Synthetic oligonucleotide
ngcacnnnggagngancggtt

SEQ ID NO: 18 - DNA, 21 bp, Artificial Sequence
  Description of Combined DNA/RNA Molecule Synthetic oligonucleotide
ccgancacnccaaagngcatt

SEQ ID NO: 19 - DNA, 21 bp, Artificial Sequence
  Description of Combined DNA/RNA Molecule Synthetic oligonucleotide
gagncccgggaagccccagtt

SEQ ID NO: 20 - DNA, 21 bp, Artificial Sequence
  Description of Combined DNA/RNA Molecule Synthetic oligonucleotide
cnggggcnncccgggacnctt

SEQ ID NO: 21 - DNA, 21 bp, Artificial Sequence
  Description of Combined DNA/RNA Molecule Synthetic oligonucleotide
aaaggaaccnacnngnacatt

SEQ ID NO: 22 - DNA, 21 bp, Artificial Sequence
  Description of Combined DNA/RNA Molecule Synthetic oligonucleotide
ngnacaagnaggnnccnnntt

SEQ ID NO: 23 - DNA, 21 bp, Artificial Sequence
  Description of Combined DNA/RNA Molecule Synthetic oligonucleotide
uccucagccucuucuccuutt

SEQ ID NO: 24 - DNA, 21 bp, Artificial Sequence
  Description of Combined DNA/RNA Molecule Synthetic oligonucleotide
aaggagaagaggcugaggatt

SEQ ID NO: 25 - DNA, 19 bp, Artificial Sequence
  Description of Combined DNA/RNA Molecule Synthetic oligonucleotide
uccucagccucuucuccuu

SEQ ID NO: 26 - DNA, 19 bp, Artificial Sequence
  Description of Combined DNA/RNA Molecule Synthetic oligonucleotide
aaggagaagaggcugagga

SEQ ID NO: 27 - DNA, 21 bp, Artificial Sequence
  Description of Combined DNA/RNA Molecule Synthetic oligonucleotide
uccucagccucuucuccuutt

SEQ ID NO: 28 - DNA, 21 bp, Artificial Sequence
  Description of Combined DNA/RNA Molecule Synthetic oligonucleotide
aaggagaagaggcugaggatt

SEQ ID NO: 29 - DNA, 21 bp, Artificial Sequence
  Description of Combined DNA/RNA Molecule Synthetic oligonucleotide
uccucagccucuucuccuutt

SEQ ID NO: 30 - DNA, 21 bp, Artificial Sequence
  Description of Combined DNA/RNA Molecule Synthetic oligonucleotide
aaggagaagaggcugaggatt

SEQ ID NO: 31 - DNA, 21 bp, Artificial Sequence
  Description of Combined DNA/RNA Molecule Synthetic oligonucleotide
uccucagccucuucuccuutt

SEQ ID NO: 32 - DNA, 21 bp, Artificial Sequence
  Description of Combined DNA/RNA Molecule Synthetic oligonucleotide
aaggagaagaggcugaggatt

SEQ ID NO: 33 - DNA, 21 bp, Artificial Sequence
  Description of Combined DNA/RNA Molecule Synthetic oligonucleotide
nccncagccncnncnccuutt

SEQ ID NO: 34 - DNA, 21 bp, Artificial Sequence
  Description of Combined DNA/RNA Molecule Synthetic oligonucleotide
aaggagaagaggcngaggatt

SEQ ID NO: 35 - DNA, 21 bp, Artificial Sequence
  Description of Combined DNA/RNA Molecule Synthetic oligonucleotide
uccncagccncnncnccuutt

SEQ ID NO: 36 - DNA, 21 bp, Artificial Sequence
  Description of Combined DNA/RNA Molecule Synthetic oligonucleotide
aaggagaagaggcngaggatt

SEQ ID NO: 37 - DNA, 21 bp, Artificial Sequence
  Description of Combined DNA/RNA Molecule Synthetic oligonucleotide
uccncagccncnncnccnntt

SEQ ID NO: 38 - DNA, 21 bp, Artificial Sequence
  Description of Combined DNA/RNA Molecule Synthetic oligonucleotide
aaggagaagaggcngaggatt

SEQ ID NO: 39 - DNA, 21 bp, Artificial Sequence
  Description of Combined DNA/RNA Molecule Synthetic oligonucleotide
uccucagccucuucuccuutt

SEQ ID NO: 40 - DNA, 21 bp, Artificial Sequence
  Description of Combined DNA/RNA Molecule Synthetic oligonucleotide
aaggagaagaggcugaggatt

SEQ ID NO: 41 - DNA, 21 bp, Artificial Sequence
  Description of Combined DNA/RNA Molecule Synthetic oligonucleotide
uccncagccncnncnccnntt

SEQ ID NO: 42 - DNA, 21 bp, Artificial Sequence
  Description of Combined DNA/RNA Molecule Synthetic oligonucleotide
aaggagaagaggcngaggatt

SEQ ID NO: 43 - DNA, 21 bp, Artificial Sequence
  Description of Combined DNA/RNA Molecule Synthetic oligonucleotide
uccucagccucuucuccuutt

SEQ ID NO: 44 - DNA, 21 bp, Artificial Sequence
  Description of Combined DNA/RNA Molecule Synthetic oligonucleotide
aaggagaagaggcugaggatt

SEQ ID NO: 45 - DNA, 21 bp, Artificial Sequence
  Description of Combined DNA/RNA Molecule Synthetic oligonucleotide
uccucagccucuucuccuutt

SEQ ID NO: 46 - DNA, 21 bp, Artificial Sequence
  Description of Combined DNA/RNA Molecule Synthetic oligonucleotide
aaggagaagaggcugaggatt

SEQ ID NO: 47 - DNA, 21 bp, Artificial Sequence
  Description of Combined DNA/RNA Molecule Synthetic oligonucleotide
uccucagccucuucuccuutt

SEQ ID NO: 48 - DNA, 21 bp, Artificial Sequence
  Description of Combined DNA/RNA Molecule Synthetic oligonucleotide
aaggagaagaggcugaggatt

SEQ ID NO: 49 - DNA, 21 bp, Artificial Sequence
  Description of Combined DNA/RNA Molecule Synthetic oligonucleotide
gggucggaacccaagcuuatt

SEQ ID NO: 50 - DNA, 21 bp, Artificial Sequence
  Description of Combined DNA/RNA Molecule Synthetic oligonucleotide
uaagcuuggguuccgacccta

SEQ ID NO: 51 - DNA, 19 bp, Artificial Sequence
  Description of Combined DNA/RNA Molecule Synthetic oligonucleotide
gggucggaacccaagcuua

SEQ ID NO: 52 - DNA, 19 bp, Artificial Sequence
  Description of Combined DNA/RNA Molecule Synthetic oligonucleotide
uaagcuuggguuccgaccc

SEQ ID NO: 53 - DNA, 19 bp, Artificial Sequence
  Description of Combined DNA/RNA Molecule Synthetic oligonucleotide
gggucggaacccaagcuua

SEQ ID NO: 54 - DNA, 21 bp, Artificial Sequence
  Description of Combined DNA/RNA Molecule Synthetic oligonucleotide
uaagcuuggguuccgacccuu

SEQ ID NO: 55 - DNA, 21 bp, Artificial Sequence
  Description of Combined DNA/RNA Molecule Synthetic oligonucleotide
gggucggaacccaagcuuatt

SEQ ID NO: 56 - DNA, 21 bp, Artificial Sequence
  Description of Combined DNA/RNA Molecule Synthetic oligonucleotide
uaagcuuggguuccgacccta

SEQ ID NO: 57 - DNA, 21 bp, Artificial Sequence
  Description of Combined DNA/RNA Molecule Synthetic oligonucleotide
gggucggaacccaagcuuatt

SEQ ID NO: 58 - DNA, 21 bp, Artificial Sequence
  Description of Combined DNA/RNA Molecule Synthetic oligonucleotide
uaagcuuggguuccgacccta

SEQ ID NO: 59 - DNA, 21 bp, Artificial Sequence
  Description of Combined DNA/RNA Molecule Synthetic oligonucleotide
gggncggaacccaagcnuatt

SEQ ID NO: 60 - DNA, 21 bp, Artificial Sequence
  Description of Combined DNA/RNA Molecule Synthetic oligonucleotide
naagcnngggnnccgacccta

SEQ ID NO: 61 - DNA, 21 bp, Artificial Sequence
  Description of Combined DNA/RNA Molecule Synthetic oligonucleotide
gggncggaacccaagcnnatt

SEQ ID NO: 62 - DNA, 21 bp, Artificial Sequence
  Description of Combined DNA/RNA Molecule Synthetic oligonucleotide
uaagcnngggnnccgacccta

SEQ ID NO: 63 - DNA, 21 bp, Artificial Sequence
  Description of Combined DNA/RNA Molecule Synthetic oligonucleotide
gggucggaacccaagcuuatt

SEQ ID NO: 64 - DNA, 21 bp, Artificial Sequence
  Description of Combined DNA/RNA Molecule Synthetic oligonucleotide
uaagcuuggguuccgacccta

SEQ ID NO: 65 - DNA, 21 bp, Artificial Sequence
  Description of Combined DNA/RNA Molecule Synthetic oligonucleotide
gggucggaacccaagcuuatt

SEQ ID NO: 66 - DNA, 21 bp, Artificial Sequence
  Description of Combined DNA/RNA Molecule Synthetic oligonucleotide
uaagcuuggguuccgacccta

SEQ ID NO: 67 - DNA, 21 bp, Artificial Sequence
  Description of Combined DNA/RNA Molecule Synthetic oligonucleotide
gggncggaacccaagcnuatt

SEQ ID NO: 68 - DNA, 20 bp, Artificial Sequence
  Description of Combined DNA/RNA Molecule Synthetic oligonucleotide
nagcnngggnnccgacccta

SEQ ID NO: 69 - DNA, 21 bp, Artificial Sequence
  Description of Combined DNA/RNA Molecule Synthetic oligonucleotide
gggncggaacccaagcnnatt

SEQ ID NO: 70 - DNA, 21 bp, Artificial Sequence
  Description of Combined DNA/RNA Molecule Synthetic oligonucleotide
uaagcnngggnnccgacccta

SEQ ID NO: 71 - DNA, 21 bp, Artificial Sequence
  Description of Combined DNA/RNA Molecule Synthetic oligonucleotide
gggucggaacccaagcuuatt

SEQ ID NO: 72 - DNA, 21 bp, Artificial Sequence
  Description of Combined DNA/RNA Molecule Synthetic oligonucleotide
uaagcuuggguuccgacccta

SEQ ID NO: 73 - DNA, 21 bp, Artificial Sequence
  Description of Combined DNA/RNA Molecule Synthetic oligonucleotide
gggncggaacccaagcnuatt

SEQ ID NO: 74 - DNA, 19 bp, Artificial Sequence
  Description of Combined DNA/RNA Molecule Synthetic oligonucleotide
uaagcuuggguuccgaccc

SEQ ID NO: 75 - DNA, 21 bp, Artificial Sequence
  Description of Combined DNA/RNA Molecule Synthetic oligonucleotide
gggncggaacccaagcnuatt

SEQ ID NO: 76 - DNA, 19 bp, Artificial Sequence
  Description of Combined DNA/RNA Molecule Synthetic oligonucleotide
uaagcuuggguuccgaccc

SEQ ID NO: 77 - DNA, 21 bp, Artificial Sequence
  Description of Combined DNA/RNA Molecule Synthetic oligonucleotide
cuacacaaaucagcgauuutt

SEQ ID NO: 78 - DNA, 21 bp, Artificial Sequence
  Description of Combined DNA/RNA Molecule Synthetic oligonucleotide
aaaucgcugauuuguguagtt

SEQ ID NO: 79 - DNA, 21 bp, Artificial Sequence
  Description of Combined DNA/RNA Molecule Synthetic oligonucleotide
cnacacaaancagcgannntt

SEQ ID NO: 80 - DNA, 21 bp, Artificial Sequence
  Description of Combined DNA/RNA Molecule Synthetic oligonucleotide
aaancgcngannngngnagtt

SEQ ID NO: 81 - DNA, 21 bp, Artificial Sequence
  Description of Combined DNA/RNA Molecule Synthetic oligonucleotide
cuacacaaaucagcgauuutt

SEQ ID NO: 82 - DNA, 21 bp, Artificial Sequence
  Description of Combined DNA/RNA Molecule Synthetic oligonucleotide
aaancgcngannngngnagtt

SEQ ID NO: 83 - DNA, 21 bp, Artificial Sequence
  Description of Combined DNA/RNA Molecule Synthetic oligonucleotide
cnacacaaancagcgannntt

SEQ ID NO: 84 - DNA, 21 bp, Artificial Sequence
  Description of Combined DNA/RNA Molecule Synthetic oligonucleotide
aaaucgcugauuuguguagtt

(19) United States
(12) Patent Application Publication
(10) Pub. No.: US 2006/0160123 A1
(43) Pub. Date: Jul. 20, 2006

(54) Method of minimizing off-target effects of siRNA molecules

(75) Inventor: Steven C. Quay, Seattle, WA (US)

Correspondence Address: NASTECH PHARMACEUTICAL COMPANY INC, 3450 MONTE VILLA PARKWAY, BOTHELL, WA, 98021-8906, US

(73) Assignee: Nastech Pharmaceutical Company Inc.

(21) Appl. No.: 11/361,793

(22) Filed: Feb. 24, 2006

Related U.S. Application Data
(63) Continuation of application No. 11/219,625, filed on Sep. 2, 2005.
(63) Continuation-in-part of application No. 10/925,314, filed on Aug. 24, 2004.
(60) Provisional application No. 60/497,740, filed on Aug. 25, 2003.

(51) Int. Cl. C12Q 1/68; A61K 48/00

(52) U.S. Cl. 435/6; 514/44

(57) ABSTRACT

The invention relates to a method of minimizing off-target effects of siRNA, comprising preparing double-stranded RNA (dsRNA) having a sense strand that is homologous to a sequence of a target gene and an anti-sense strand that is complementary to said sense strand, and having at least one pyrimidine replaced by a 5′-methyl-pyrimidine, and contacting said dsRNA with a cell capable of expressing said target gene.

This patent application is a continuation of U.S. patent application Ser. No. 11/219,625 filed Sep. 2, 2005, and a continuation-in-part application of U.S. patent application Ser. No. 10/925,314, filed Aug. 24, 2004, which claims priority under 35 U.S. §119 (e) of U.S. Provisional Application No. 60/497,740 filed Aug. 25, 2003 the contents of which are incorporated herein by reference.

### BACKGROUND OF THE INVENTION

RNA interference refers to the process of sequence-specific post-transcriptional gene silencing in animals mediated by short interfering RNAs (siRNAs). See Fire et al, Nature, 391:806 (1998) and Hamilton et al., Science, 286: 950-951 (1999). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla [Fire et al., Trends Genet. 15: 358 (1999)]. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. The presence of dsRNA in cells triggers the RNAi response though a mechanism that has yet to be fully characterized. This mechanism appears to be different from the interferon response that results from dsRNA-mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNAs) [Hamilton et al., supra; Berstein et al., Nature, 409: 363(2001)]. Short interfering RNAs derived from dicer activity are typically about 21 to about 23 nucleotides in length and comprise about 19 base pair duplexes [Hamilton et al., supra; Elbashir et al., Genes Dev., 15: 188 (2001)]. Dicer has also been implicated in the excision of 21- and 22-nucleotide small temporal RNAs (stRNAs) from precursor RNA of conserved structure that are implicated in translational control [Hutvagner et al., Science, 293: 834 (2001)]. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex [Elbashir et al., 2001, Genes Dev., 15, 188 (2001)].

RNAi has been studied in a variety of systems. Fire et al., Nature, 391: 806 (1998), were the first to observe RNAi in C. elegans. Bahramian and Zarbl, Molecular and Cellular Biology, 19: 274-283 (1999) and Wianny and Goetz, 1999, Nature Cell Biol., 2, 70, describe RNAi mediated by dsRNA in mammalian systems. Hammond et al., Nature, 404: 293 (2000), describe RNAi in Drosophila cells transfected with dsRNA. Elbashir et al., Nature, 411: 494 (2001), describe RNAi induced by introduction of duplexes of synthetic 21-nucleotide RNAs in cultured mammalian cells including human embryonic kidney and HeLa cells. Recent work in Drosophila embryonic lysates [Elbashir et al., EMBO J, 20: 6877 (2001)] has revealed certain requirements for siRNA length, structure, chemical composition, and sequence that are essential to mediate efficient RNAi activity. These studies have shown that 21-nucleotide siRNA duplexes are most active when containing 3′-terminal dinucleotide overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with 2′-deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end of the guide sequence (Elbashir et al., EMBO J, 20: 6877 (2001)]. Other studies have indicated that a 5′-phosphate on the target-complementary strand of a siRNA duplex is required for siRNA activity and that ATP is utilized to maintain the 5′-phosphate moiety on the siRNA (Nykanen et al., Cell, 107: 309 (2001)].

Recent developments in the areas of gene therapy, antisense therapy and RNA interference therapy have created a need to develop efficient means of introducing nucleic acids into cells. Unfortunately, existing techniques for delivering nucleic acids to cells are limited by instability of the nucleic acids, poor efficiency and/or high toxicity of the delivery reagents.

Thus, there is a need to provide for methods and compositions for effectively delivering double-stranded nucleic acids to cells to produce an effective therapy especially for delivering siRNAs for RNA interference therapy.

### SUMMARY OF THE INVENTION

One aspect of the invention is a double-stranded RNA (dsRNA) molecule comprising between about 15 base pairs and about 40 base pairs, in which at least one ribonucleotide of the dsRNA is a 5′-methyl-pyrimidine, preferably a ribothymidine. In a preferred embodiment the dsRNA molecule is an siRNA molecule comprising a sense strand that is homologous to a sequence of a target gene and an anti-sense strand that is complementary to said sense strand, and in which at least one uridine of the siRNA sequence is replaced by a ribothymidine. In an alternate embodiment, at least three of the uridines of the siRNA sequence are replaced by ribothymidines. In other alternate embodiments, all of the uridines of the sense strand of the siRNA sequence are replaced by ribothymidines, or all of the uridines of the antisense strand of the siRNA sequence are replaced by ribothymidines, or all of the uridines in the siRNA sequence are replaced by ribothymidines. The dsRNA molecule may have a 3′ overhang or may be blunt ended.

In another aspect of the invention, the replacement of uridine by ribothymidine in the dsRNA molecule improves ribonuclease stability to the dsRNA when the dsRNA is contacted with a biological sample, e.g., blood serum or plasma.

Another aspect of the invention is the replacement of uridine by ribothymidine in the dsRNA molecule to reduce interferon responsiveness of the siRNA molecule when the siRNA is contacted with a biological cell.

Another aspect of the invention is a method of minimizing off-target effects of siRNA, comprising preparing double-stranded RNA (dsRNA) having a sense strand that is homologous to a sequence of a target gene and an anti-sense strand that is complementary to said sense strand, and having at least one pyrimidine replaced by a 5′-methyl-pyrimidine, and contacting said dsRNA with a cell capable of expressing said target gene. In one embodiment, the 5′-methyl-pyrimidine is ribothymidine. In a related embodiment, at least three of the uridines of the siRNA sequence are replaced by ribothymidines. In another embodiment, at least three of the uridines of the sense strand of the siRNA sequence are replaced by ribothymidines. In another embodiment at least three of the uridines of the antisense strand of the siRNA sequence are replaced by ribothymidines. In another embodiment, all of the uridines in the siRNA sequence are replaced by ribothymidines. In another embodiment, the siRNA molecule has a 3′ overhang or, alternatively, is blunt ended.

Another aspect of the invention is a method of increasing stability of siRNA, comprising preparing double-stranded RNA (dsRNA) having a sense strand that is homologous to a sequence of a target gene and an anti-sense strand that is complementary to said sense strand, and having at least one pyrimidine replaced by a 5′-methyl-pyrimidine, and contacting said dsRNA with a biological sample. In one embodiment, the biological sample is blood serum or plasma. In another embodiment, at least three of the uridines of the sense strand of the siRNA sequence are replaced by ribothymidines. In another embodiment at least three of the uridines of the antisense strand of the siRNA sequence are replaced by ribothymidines. In another embodiment, all of the uridines in the siRNA sequence are replaced by ribothymidines.

### BRIEF DESCRIPTION OF THE DRAWING

FIG. 1 is an SDS PAGE gel showing the results of the stability studies of Example 3, in which the stable siRNA construct in which all of the uridines are changed to 5-methyluridine ribothymidine.

### DESCRIPTION OF THE INVENTION

The present invention also features a method for preparing the claimed ds RNA nanoparticles. A first solution containing one of the melamine derivatives disclosed above is dissolved in an organic solvent such as dimethyl sulfoxide, or dimethyl formamide to which an acid such as HCl has been added. The concentration of HCl would be about 3.3 moles of HCl for every mole of the melamine derivative. The first solution is then mixed with a second solution, which includes a nucleic acid dissolved or suspended in a polar or hydrophilic solvent (e.g., an aqueous buffer solution containing, for instance, ethylenediaminetraacetic acid (EDTA), or tris(hydroxymethyl) aminomethane (TRIS), or combinations thereof. The mixture forms a first emulsion. The mixing can be done using any standard technique such as, for example sonication, vortexing, or in a microfluidizer. This causes complexing of the nucleic acids with the melamine derivative forming a trimeric nucleic acid complex. While not being bound to theory or mechanism, it is believed that three nucleic acids are complexed in a circular fashion about one melamine derivative moiety, and that a number of the melamine derivative moieties can be complexed with the three nucleic acid molecules depending on the size of the number of nucleotides that the nucleic acid has. The concentration should be at least 1 to 7 moles of the melamine derivative for every mole of a double stranded nucleic acid having 20 nucleotide pairs, more if the ds nucleic acid is larger. The resultant nucleic acid particles can be purified and the organic solvent removed using size-exclusion chromatography or dialysis or both.

The complexed nucleic acid nanoparticles can then be mixed with an aqueous solution containing either polyarginine, a Gln-Asn polymer, or both in an aqueous solution. The preferred molecular weight of each polymer is 5000-15,000 Daltons. This forms a solution containing nanoparticles of nucleic acid complexed with the melamine derivative and the polyarginine and the Gln-Asn polymers. The mixing steps are carried out in a manner that minimizes shearing of the nucleic acid while producing nanoparticles on average smaller than 200 nanometers in diameter. While not being bound by theory of mechanism, it is believed that the polyarginine complexes with the negative charge of the phosphate groups within the minor groove of the nucleic acid, and the polyarginine wraps around the trimeric nucleic acid complex. At either terminus of the polyarginine other moieties, such as the TAT polypeptide, mannose or galactose, can be covalently bound to the polymer to direct binding of the nucleic acid complex to specific tissues, such as to the liver when galactose is used. While not being bound to theory, it is believed that the Gln-Asn polymer complexes with the nucleic acid complex within the major groove of the nucleic acid through hydrogen bonding with the bases of the nucleic acid. The polyarginine and the Gln-Asn polymer should be present at a concentration of 2 moles per every mole of nucleic acid having 20 base pairs. The concentration should be increased proportionally for a nucleic acid having more than 20 base pairs. So perhaps, if the nucleic acid has 25 base pairs, the concentration of the polymers should be 2.5-3 moles per mole of ds nucleic acid. An example of is a polypeptide operatively linked to an N-terminal protein transduction domain from HIV TAT. The HIV TAT construct for use in such a protein is described in detail in Vocero-Akbani et al. Nature Med., 5:23-33 (1999). See also United States Patent Application No. 20040132161, published on Jul. 8, 2004.

The resultant nanoparticles can be purified by standard means such as size exclusion chromatography followed by dialysis. The purified complexed nanoparticles can then be lyophilized using techniques well known in the art.

This method of delivering double-stranded nucleic acids is especially useful in the context of therapeutics utilizing RNA interference. RNA interference or RNAi is a system in most plant and animal cells that censors the expression of genes. The genes might be the genes of the host cell that is being inappropriately expressed or viral nucleic acids. When a threatening gene is expressed, the RNAi machinery silences it by intercepting and destroying only the offending messenger RNA (mRNA), without disturbing the mRNA expressed from other genes.

Scientists have now discovered how to synthetically produce double-stranded RNA that is able to trigger the RNAi machinery to destroy a desired mRNA. The scientist produces a short antisense strand (generally 30 base pairs or less) and a sense strand that hybridizes to the antisense strand. This short dsRNA is called a short (or small) interfering RNA, or siRNA. The antisense strand is a stretch of RNA that specifically binds to an mRNA that the scientist wishes to silence. When an siRNA is inserted into a cell, the siRNA duplex is then unwound, and the antisense strand of the duplex is loaded into an assembly of proteins to form the RNA-induced silencing complex (RISC).

Within the silencing complex, the siRNA molecule is positioned so that mRNAs can bump into it. The RISC will encounter thousands of different mRNAs that are in a typical cell at any given moment. But the siRNA of the RISC will adhere well only to an mRNA that closely complements its own nucleotide sequence. So unlike an interferon response to a viral infection, the silencing complex is highly selective in choosing its target mRNAs.

When a matched mRNA finally docks onto the siRNA, an enzyme know as slicer cuts the captured mRNA strand in two. The RISC then releases the two pieces of the mRNA (now rendered incapable of directing protein synthesis) and moves on. The RISC itself stays intact capable of finding and cleaving another mRNA.

A preferred embodiment of the present invention is comprised of nanoparticles of double-stranded RNA less than 100 nanometers (nm). More, specifically, the double-stranded RNA is less than about 30 nucleotide pairs in length, preferably 20-25 nucleotide base pairs in length. More specifically, the present invention is comprised of a double-stranded RNA complex wherein two or more double-stranded

In a preferred embodiment, the ribose uracils of the siRNA are replaced with ribose thymine. In fact it has been surprisingly discovered that the stability of double-stranded RNA is greatly increased and is less susceptible to degradation by Rnases when all of the ribose uracils are change to ribose thymine in both the sense and anti-sense strands of the RNA. Thus a preferred siRNA is a double-stranded RNA having 15-30 bases pairs wherein all of the ribose uracils that would normally be present have been changed to a 5-alkyluridine such as ribothymidine (rT) [5-methyluridine]. Alternatively, some of the uracils can be changed so that only those ribose uracils present in the sense strand are changed to ribothymidine, or in the alternative, only those ribose uracils present in the antisense strand are changed to ribothymidine. Examples 2 and 3 illustrate this aspect of the invention.

For example a stable siNA duplex of the present invention which would target the mRNA of the VEGF receptor 1 (see SEQ ID NO:2000 of United States Patent Application Publication No. 2004/01381 published Jul. 15, 2004 would be:

(SEQ ID NO:9)

G.C.A.rT.rT.rT.G.G.C.A.rT.A.A.G.A.A.A.rTdTdT

(SEQ ID NO:10)

A.rT.rT.rTrT.C.rT.rT.A.rT.G.C.C.A.A.A.rT.C.dT.dT

An siNA duplex of the present invention, which would target the RNA of Hepatitis B virus and target a subsequence of the HBV RNA would be:

(SEQ ID NO:11)

C.C.rT.G.C.rT.G.C.rT.A.rT.G.C.C.rT.C.A.rT.C.dT.dT

(SEQ ID NO:12)

G.A.rT.G.A.G.G.C,A.rT.A.G.C.A.G.C.A.G.G.dTdT

See United States Patent Application Publication No. 2003/0206887 published Nov. 6, 2003.

An siNA duplex of the present invention which would target RNA of the human immunodeficiency virus (HIV) would be:

(SEQ ID NO:13)

rT.rT.rT.G.G.A.A.A.G.G.A.C.C.A.G.C.A.A.A.dT.dT

(SEQ ID NO:14)

rT.rT.rT.G.C.rT.G.G.rT.C.C.rTrT.rT.C.C.A.A.A.dT.dT

See United States Patent Application Publication No. 2003/0175950 published Sep. 18, 2003.

An siNA duplex of the present invention which would target the mRNA of human tumor necrosis factor-alpha (TNFα) would be:

(SEQ ID NO:15)

C.A.C.C.C.rT.G.A.C.A.A.G.C.rT.G.C.C.A.G.dT.dT

(SEQ ID NO:16)

C.rT.G.G.C.A.G.C.rT.rT.G.rT.C.A.G.G.G.rT.G.dT.dT

Another siNA targeted against the TNFα mRNA would be:

(SEQ ID NO:17)

rT.G.C.A.C.rT.rT.rT.G.G.A.G.rT.G.A.rT.C.G.G.dT.dT

(SEQ ID NO:18)

C.C.G.A.rT.C.A.C.rT.C.C.A.A.A.G.rT.G.C.A.dT.dT

An siNA duplex of the present invention targeted against the TNFα-receptor 1A mRNA would be:

(SEQ ID NO:19)

G.A.G.rT.C.C.C.G.G.G.A.A.G.C.C.C.C.A.G.dT.dT

(SEQ ID NO:20)

C.rT.G.G.G.G.C.rTrT.C.C.C.G.G.G.A.C.rT.C.dT.dT

Another siNA duplex of the present invention targeted against the TNFα-receptor 1A mRNA would be:

(SEQ ID NO:21)

A.A.A.G.G.A.A.C.C.rT.A.C.rT.rT.G.rT.A.C.A.dT.dT

(SEQ ID NO:22)

rT.G.rT.A.C.A.A.G.rT.A.G.G.rT.rT.C.C.rT.rT.rT.dT.d
T

See International Patent Application Publication No. WO 03/070897. ‘RNA Interference Mediated Inhibition of TNF and TNF Receptor Superfamily Gene Expression Using Short Interfering Nucleic Acid (siNA)’. These would be useful in treating TNF-α associated diseases as rheumatoid arthritis.

As used herein “cell” is used in its usual biological sense, and does not refer to an entire multicellular organism, e.g., specifically does not refer to a human. The cell can be present in an organism, e.g., birds, plants and mammals such as humans, cows, sheep, apes, monkeys, swine, dogs, and cats. The cell can be prokaryotic (e.g., bacterial cell) or eukaryotic (e.g., mammalian or plant cell). The cell can be of somatic or germ line origin, totipotent or pluripotent, dividing or non-dividing. The cell can also be derived from or can comprise a gamete or embryo, a stem cell, or a fully differentiated cell.

In another aspect, the invention provides mammalian cells containing one or more siNA molecules of this invention. The one or more siNA molecules can independently be targeted to the same or different sites.

By “RNA” is meant a molecule comprising at least one ribonucleotide residue. By “ribonucleotide” is meant a nucleotide with a hydroxyl group at the 2′ position of a .beta.-D-ribo-furanose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

By “subject” is meant an organism, which is a donor or recipient of explanted cells or the cells themselves. “Subject” also refers to an organism to which the nucleic acid molecules of the invention can be administered. In one embodiment, a subject is a mammal or mammalian cells. In another embodiment, a subject is a human or human cells.

The term “universal base” as used herein refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example Loakes, 2001, Nucleic Acids Research, 29, 2437-2447).

The nucleic acid molecules of the instant invention, individually, or in combination or in conjunction with other drugs, can be used to treat diseases or conditions discussed herein. For example, to treat a particular disease or condition, the siNA molecules can be administered to a patient or can be administered to other appropriate cells evident to those skilled in the art, individually or in combination with one or more drugs under conditions suitable for the treatment.

In a further embodiment, the siNA molecules can be used in combination with other known treatments to treat conditions or diseases discussed above. For example, the described molecules could be used in combination with one or more known therapeutic agents to treat a disease or condition. Non-limiting examples of other therapeutic agents that can be readily combined with a siNA molecule of the invention are enzymatic nucleic acid molecules, allosteric nucleic acid molecules, antisense, decoy, or aptamer nucleic acid molecules, antibodies such as monoclonal antibodies, small molecules, and other organic and/or inorganic compounds including metals, salts and ions.

By “comprising” is meant including, but not limited to, whatever follows the word “comprising.” Thus, use of the term “comprising” indicates that the listed elements are required or mandatory, but that other elements are optional and may or may not be present. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small” refers to nucleic acid motifs no more than 100 nucleotides in length, preferably no more than 80 nucleotides in length, and most preferably no more than 50 nucleotides in length; e.g., individual siNA oligonucleotide sequences or siNA sequences synthesized in tandem) are preferably used for exogenous delivery. The simple structure of these molecules increases the ability of the nucleic acid to invade targeted regions of protein and/or RNA structure. Exemplary molecules of the instant invention are chemically synthesized, and others can similarly be synthesized.

Oligonucleotides (e.g., certain modified oligonucleotides or portions of oligonucleotides lacking ribonucleotides) are synthesized using protocols known in the art, for example as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. RNA including certain siNA molecules of the invention follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59.

Alternatively, the nucleic acid molecules of the present invention can be synthesized separately and joined together post-synthetically, for example, by ligation (Moore et al., 1992, Science 256, 9923; Draper et al., International PCT Publication No. WO 93/23569; Shabarova et al., 1991, Nucleic Acids Research 19, 4247; Bellon et al., Nucleosides & Nucleotides, 16, 951 (1997); Bellon et al., Bioconjugate Chem. 8, 204 (1997), or by hybridization following synthesis and/or deprotection.

Administration of Nucleic Acid Molecules

Methods for the delivery of nucleic acid molecules are described in Akhtar et al., Trends Cell Bio., 2, 139 (1992); Delivery Strategies for Antisense Oligonucleotide Therapeutics, ed. Akhtar, 1995, Maurer et al., Mol. Membr. Biol., 16: 129-140 (1999); Hofland and Huang, Handb. Exp. Pharmacol., 137: 165-192 (1999); and Lee et al., ACS Symp. Ser., 752: 184-192 (2000), Sullivan et al., PCT WO 94/02595, further describes the general methods for delivery of enzymatic nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as hydrogels, cyclodextrins, biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., Clin. Cancer Res., 5: 2330-2337 (1999) and Barry et al., International PCT Publication No. WO 99/31262. The molecules of the instant invention can be used as pharmaceutical agents. Pharmaceutical agents prevent, modulate the occurrence, or treat (alleviate a symptom to some extent, preferably all of the symptoms) of a disease state in a patient.

Thus, the invention features a pharmaceutical composition comprising one or more nucleic acid(s) of the invention in an acceptable carrier, such as a stabilizer, buffer, and the like. The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity and forms that prevent the composition or formulation from exerting its effect.

By “systemic administration” is meant in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

By “pharmaceutically acceptable formulation” is meant, a composition or formulation that allows for the effective distribution of the nucleic acid molecules of the instant invention in the physical location most suitable for their desired activity. Nonlimiting examples of agents suitable for formulation with the nucleic acid molecules of the instant invention include: P-glycoprotein inhibitors (such as Pluronic P85), which can enhance entry of drugs into the CNS [Jolliet-Riant and Tillement, Fundam. Clin. Pharmacol., 13:16-26 (1999)]; biodegradable polymers, such as poly (DL-lactide-coglycolide) microspheres for sustained release delivery after intracerebral implantation (Emerich, D F et al., Cell Transplant, 8: 47-58 (1999)] (Alkermes, Inc. Cambridge, Mass.); and loaded nanoparticles, such as those made of polybutylcyanoacrylate, which can deliver drugs across the blood brain barrier and can alter neuronal uptake mechanisms (Prog Neuropsychopharmacol Biol Psychiatry, 23: 941-949, (1999)]. Other non-limiting examples of delivery strategies for the nucleic acid molecules of the instant invention include material described in Boado et al., J. Pharm. Sci., 87:1308-1315 (1998); Tyler et al., FEBS Lett., 421: 280-284 (1999); Pardridge et al., PNAS USA., 92: 5592-5596 (1995); Boado, Adv. Drug Delivery Rev., 15: 73-107 (1995); Aldrian-Herrada et al., Nucleic Acids Res., 26: 4910-4916 (1998); and Tyler et al., PNAS USA., 96: 7053-7058 (1999).

The invention also features the use of the composition comprising surface-modified liposomes containing poly(ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev., 95:2601-2627 (1995); Ishiwata et al., Chem. Pharm. Bull., 43: 1005-1011 (1995)]. Such liposomes have been shown to accumulate selectively in tumors, presumably by extravasation and capture in the neovascularized target tissues [Lasic et al., Science, 267: 1275-1276 (1995); Oku et al., Biochim. Biophys. Acta, 1238, 86-90 (1995)]. The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 42: 24864-24870 (1995); Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also includes compositions prepared for storage or administration, which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985). For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective dose is that dose required to prevent, inhibit the occurrence of, or treat (alleviate a symptom to some extent, preferably all of the symptoms) a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The present invention also includes compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring, and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention may also be administered to a patient in combination with other therapeutic compounds to increase the overall therapeutic effect. The use of multiple compounds to treat an indication may increase the beneficial effects while reducing the presence of side effects.

In one embodiment, the invention compositions suitable for administering nucleic acid molecules of the invention to specific cell types, such as hepatocytes. For example, the asialoglycoprotein receptor (ASGPr) (Wu and Wu, J. Biol. Chem. 262:4429-4432 (1987)] is unique to hepatocytes and binds branched galactose-terminal glycoproteins, such as asialoorosomucoid (ASOR). Binding of such glycoproteins or synthetic glycoconjugates to the receptor takes place with an affinity that strongly depends on the degree of branching of the oligosaccharide chain, for example, triatennary structures are bound with greater affinity than biatenarry or monoatennary chains (Baenziger and Fiete, Cell, 22: 611-620 (1980); Connolly et al., J. Biol. Chem., 257: 939-945 (1982). Lee and Lee, Glycoconjugate J., 4: 317-328 (1987), obtained this high specificity through the use of N-acetyl-D-galactosamine as the carbohydrate moiety, which has higher affinity for the receptor, compared to galactose. This “clustering effect” has also been described for the binding and uptake of mannosyl-terminating glycoproteins or glycoconjugates (Ponpipom et al., J. Med. Chem., 24: 1388-1395(1981). The use of galactose and galactosamine based conjugates to transport exogenous compounds across cell membranes can provide a targeted delivery approach to the treatment of liver disease such as HBV infection or hepatocellular carcinoma. The use of bioconjugates can also provide a reduction in the required dose of therapeutic compounds required for treatment. Furthermore, therapeutic bioavialability, pharmacodynamics, and pharmacokinetic parameters can be modulated through the use of nucleic acid bioconjugates of the invention.

### EXAMPLE 1

### Preparation of Melamine Derivatives

### Methods and Materials for 2,4,6-Triamidosarcocyl Melamine

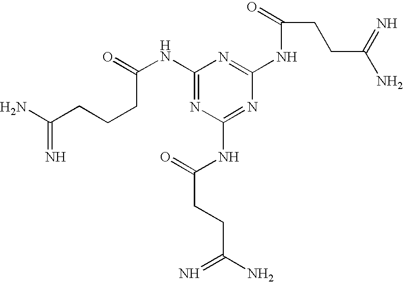

### 4-Methoxy-2,3,6-trimethylbenzenesulfonyl (Mtr) creatine

A solution of creatine (390 mgs-3 mmol) in a mixture of 4N NaOH (3 ml) and acetone is cooled in an ice water bath and treated with Mtr chloride (680 mgs-5.25 mmol) in acetone (3 mls). The mixture is stirred overnight at room temperature and then acidified with 10% citric acid in water. The acetone is evaporated and the residual aqueous suspension is extracted with ethyl acetate, 3×10 ml. The combined extracts are dried over magnesium sulfate, filtered and the filtrate is evaporated to dryness. The residue is crystallized from ethyl acetate:hexane.

### 2,4,6-Mtr-triamidosarcocyl melamine

The Mtr-creatine (694 mgs-2 mmol) is dissolved in 5 ml of dimethylformamide (DMF) with melamine (76 mgs-0.6 mmol), hydroxybenzotriazole (310 mgs-2 mmol) and diisopropylethylamine (403 ul-2.3 mmol). With the addition of diisopropylcarbodiimide (DIC) (310 ul-2 mmol) the mixture is stirred overnight at room temperature.

The next day the reaction is diluted with 50 ml of ethyl acetate, extracted 3×10 ml of 10% citric acid, 1× brine, 3×10% sodium bicarbonate and 1× brine. The ethyl acetate is dried over magnesium sulfate, filtered, evaporated and the residue is crystallized from ether:hexane.

### 2,4,6-Triamidosarcocyl Melamine

The 2,4,6-Mtr-triamidosarcocyl melamine (340 mgs-0.3 mmol) is dissolved in trifluoroacetic acid:thianisole (95:5) (5 ml) and stirred of for four hours. The solution is evaporated to an oil and triturated with ether and dried.

### Methods and Materials for 2,4,6-Triguanidino Triazine

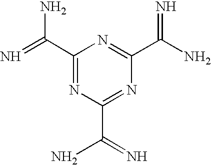

### Melamine Trithiourea Sulfonic Acid

A mixture of melamine (1620 mgs-13 mmol) is and methyl thiocynate (2870 mgs-139 mmol) in 70 mls of ethyl alcohol is refluxed for one hour. After evaporation the corresponding urea is isolated by evaporation of the alcohol. The triisothiourea triazine intermediate is then dissolved in water (10 ml) containing sodium chloride (mg-mmol), sodium molybdate dehydrate and cooled to 0° C. with vigorous stirring. Hydrogen peroxide (30%-41 mmol) is added dropwise to the stirring suspension. The sulfonic acid product is collected by filtration and washed with cold brine and dried.

### 2,4,6-Triguanidino Triazine

The melamine trithiourea sulfonic acid (1520 mgs-10 mmol) is added to the appropriate amine (13 mmol) in 5 ml of acetonitrile at room temperature. The mixture is stirred overnight. The pH is adjusted to 12 with 3N NaOH. Depending on the amine used, the guanidine product can be filtered of a s solid or extracted with methylene chloride for isolation purposes.

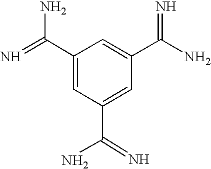

### EXAMPLE 2

### Beta-Gal siRNA Sequence

The double-stranded siRNA sequences shown below were produced synthesize using standard techniques. The siRNA sequences were designed to silence the beta galactosidase mRNA. The siRNAs were encapsulated in lipofectamine to promote transfection of the siRNA into the cells. The sequences are identical except for the varied substitution of ribose uracils by ribose thymines. The siRNA of duplex 4 did not replace any of the ribose uracils with ribose thymine. The siRNAs of duplexes 1-3 represent siRNAs of the present invention in which some or all of the uracils present in duplex 4 have been changed to ribose thymines. All of the uracils have been changed to ribose thymines in the siRNA of duplex 1. Only the uracils in the sense strand have been changed to ribose thymines in the siRNA of duplex 2. In duplex 3 only the uracils in the antisense strand were changed to ribose thymines. The purpose of the present experiment was to determine which siRNAs would be effective in silencing the β-galactosidase mRNA.

| | | |
|---|---|---|
| 1. | Duplex 1 | |
| | C.rT.A.C.A.C.A.A.A.rT.C.A.G.C.G.A. | (SEQ ID NO:1) |
| | rT.rT.rT.dT.dT | |
| | A.A.A.rT.C.G.C.rT.G.A.rT.rT.rT.G.r | (SEQ ID NO:2) |
| | T.G.rT.A.G.dT.dT | |
| 2. | Duplex 2 | |
| | C.rT.A.C.A.C.A.A.A.rT.C.A.G.C.G.A. | (SEQ ID NO:3) |
| | rT.rT.rT.dT.dT | |
| | A.A.A.U.C.G.C.U.G.A.U.U.U.G.U.G.U. | (SEQ ID NO:4) |
| | A.G.dT.dT | |
| 3. | Duplex 3 | |
| | C.U.A.C.A.C.A.A.A.U.C.A.G.C.G.A.U. | (SEQ ID NO:5) |
| | U.U.dT.dT | |
| | A.A.A.rT.C.G.C.rT.G.A.rT.rT.rT.G.r | (SEQ ID NO:6) |
| | T.G.rT.A.G.dT.dT | |
| 4. | Duplex 4 | |
| | C.U.A.C.A.C.A.A.A.U.C.A.G.C.G.A.U. | (SEQ ID NO:7) |
| | U.U.dT.dT | |
| | A.A.A.U.C.G.C.U.G.A.U.U.U.G.U.G.U. | (SEQ ID NO:8) |
| | A.G.dT.dT | |

### Procedure

### β-Gal Activity Assay Protocol for 9LacZR Cells

9lacZ/R cells were seeded in 6-well collagen-coated plates with 5×10e⁵ cells/well (2 mls total per well) and cultured with DMEM/high glucose media at 37° C. and 5% CO₂ overnight.

Preparation for transfection: 250 μl of Opti-MEM media without serum was mixed with 5 μl of 20 pmol/μl siRNA and 5 μl of Lipofectamine is mixed with another 250 μl Opti-MEM media. After both mixtures were allowed to equilibrate for 5 min, tubes were then mixed and left at room temperature for 20 min to form transfection complexes. During this time, complete media was aspirated from 6 well plates and cells were washed with incomplete Opti-MEM. 500 μl of transfection mixture were applied to wells and cells were left at 37° C. for 4 hrs. To ensure adequate coverage cells were gently shaken or rocked during this incubation.

After 4 hr incubation, the transfection media was washed once with complete DMEM/high glucose media and then replaced with the same media. The cells were then incubated for 48 hrs at 37°, 5% CO2.

### β-Galactosidase Assay (Invitrogene Assay Kit)

Transfected cells were washed with PBS, and then harvested with 0.5 mls of trypsin/EDTA. Once the cells were detached, 1 ml of complete DMEM/high glucose was added per well and the samples were transferred to microfuge tubes. The samples were then spun at 250×g for 5 minutes and the supernatant was then removed. The cells were resuspended in 50 μl of 1× lysis buffer at 4° C. The samples were then freeze-thawed with dry ice and a 37° water bath 2 times. After freeze-thawing, the samples were centrifuged for 5 minutes at 4° C. and the supernatant was transferred to a new microcentrifuge tube.

For each sample, 1.5 and 10 μl of lysate were transferred to a fresh tube and made up each sample to a final volume of 30 μl with sterile water. Add 70 μl of ONPG and 200 μl of 1× cleavage buffer with β-mercaptoethanol and mixed briefly, then incubated samples for 30 min. at 37° C. After incubation, add 500 μl of stop buffer for a final of 800 μl. Samples were then read in disposable cuvettes at 420 nm.

### Protein

Protein concentration was determined by BCA method.

### Results

All of the siRNA were effective in silencing the P-galactosidase mRNA.

### EXAMPLE 3

### Stability of siRNA in Rat Plasma

### Purpose

The purpose of this experiment was to determine how stable the siRNAs of Example 2 were to the ribonucleases present in rat plasma.

A 20 μg aliquot of each siRNA duplex of example 2 was mixed with 200 μl of fresh rat plasma incubated at 37° C. At various time points (0, 30, 60 and 20 min), 50 μl of the mixture was taken out and immediately extracted by phenol:chloroform. SiRNAs were dried following precipitation by adding 2.5 volume of isopropanol alcohol and subsequent washing step with 70% ethanol. After dissolving in water and gel loading buffer the samples were analyzed on 20% polyacrylamide gel, containing 7 M urea and visualized by ethidium bromide staining and quantitated by densitometry.

### Results

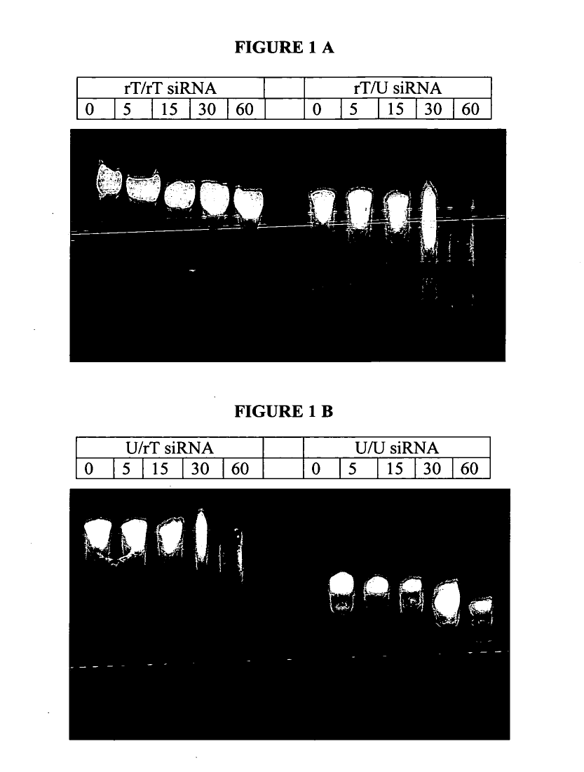

FIG. 1 shows the level of degradation at each time point for each of the constructs on a PAGE gel. Both the double strand modified (rT/rT; A) and single strand modified (U/rT and rT/U, A and B) siRNAs show little to no degradation after treatment with plasma. In contrast, the non-modified (siRNA, B) constructs begins to degrade almost immediately as indicated by the observed ladder effect on the PAGE gel. Also, the modified siRNAs have less mobility on the PAGE gel than the unmodified siRNA duplex.

Thus, it has been unexpectedly and surprisingly discovered that siRNA stability in plasma is enhanced when uridines are replaced with 5-methyluridines (ribothymidines)

### EXAMPLE 4

### Unmodified and Modified LC20 and LC13 siRNAs

Table 1 presents a list of modified and unmodified forms of LC13 siRNAs. Table 2 presents a list of modified and unmodified forms of LC20 siRNAs. The modified forms of these siRNAs include 2′-O-methyl modified ribonucleotides alone or in combination with substituting uridines with ribothymidines (5-methyluridine). A 2′-O-methyl modified ribonucleotide is indicated by a “MeO” above the ribonucleotide (e.g., N^{MeO} where N is the ribonucleotide). A ribothymidine is indicated by an “r” above the ribonucleotide (e.g., N^{r}). Each specific siRNA modification is assigned a particular label which is placed behind the LC20 and LC13 name. This allows for a direct comparison of siRNA stability and knockdown activity between two different siRNAs that have the same modification (e.g. LC13-Md15 has the same modification as LC20-MD15).

TABLE 1

| | | |
|---|---|---|
| siRNA | Nucleotide Sequence | Sequence ID |

| | | |
|---|---|---|
| LC13-WT | 5′-UCCUCAGCCUCUUCUCCUUdTdT-3′ | SEQ ID NO: 23 |
| Unmodified | 3′-dTdTAGGAGUCGGAGAAGAGGAAp-5′ | SEQ ID NO: 24 |
| LC13-19mer | 5′-UCCUCAGCCUCUUCUCCUU-3′ | SEQ ID NO: 25 |
| No 3′ | 3′-AGGAGUCGGAGAAGAGGAAp-5′ | SEQ ID NO: 26 |
| Overhangs | | |
| LC13-Md3 | 5′-UCCUCAGCCUCUUCUCCU^{MeO}U^{MeO}dTdT-3′ | SEQ ID NO: 27 |
| | 3′-dTdTA^{MeO}G^{MeO}GAGUCGGAGAAGAGGAAp-5′ | SEQ ID NO: 28 |
| LC13-Md4 | 5′-U^{MeO}C^{MeO}CUCAGCCUCUUCUCCU^{MeO}U^{MeO}dTdT-3′ | SEQ ID NO: 29 |
| | 3′-dTdTA^{MeO}G^{MeO}GAGUCGGAGAAGAGGA^{MeO}A^{MeO} p-5′ | SEQ ID NO: 30 |
| LC13-Md5 | 5′-U^{MeO}C^{MeO}CUCAGCCUCUUCUCCUUdTdT-3′ | SEQ ID NO: 31 |
| | 3′-dTdTAGGAGUCGGAGAAGAGGA^{MeO}A^{MeO}p-5′ | SEQ ID NO: 32 |
| LC13-Md6 | 5′-T^{r}CCT^{r}CAGCCT^{r}CT^{r}T^{r}CT^{r}CCU^{MeO}U^{MeO}dT dT-3′ | SEQ ID NO: 33 |
| | 3′-dTdTA^{MeO}G^{MeO}GAGT^{r}CGGAGAAGAGGAA p-5′ | SEQ ID NO: 34 |
| LC13-Md7 | 5′-U^{MeO}C^{MeO}CT^{r}CAGCCT^{r}CT^{r}T^{r}CT^{r}CCU^{MeO}U^{MeO}dTdT-3′ | SEQ ID NO: 35 |
| | 3′-dTdTA^{MeO}G^{MeO}GAGT^{r}CGGAGAAGAGGA^{MeO}A^{MeO} p-5′ | SEQ ID NO: 36 |
| LC13-Md8 | 5′-U^{MeO}C^{MeO}CT^{r}GAGCCT^{r}CT^{r}T^{r}CT^{r}CCT^{r}T^{r}dTdT-3′ | SEQ ID NO: 37 |
| | 3′-dTdTAGGAGT^{r}CGGAGAAGAGGA^{MeO}A^{MeO} p-5′ | SEQ ID NO: 38 |
| LC13-Md12 | 5′-UCCUCAGCCUCUUCUCCUU^{MeO}dT dT-3′ | SEQ ID NO: 39 |
| | 3′-dTdTA^{MeO}GGAGUCGGAGAAGAGGA Ap-5′ | SEQ ID NO: 40 |
| LC13-Md13 | 5′-U^{MeO}CCT^{r}CAGCCT^{r}CT^{r}T^{r}CT^{r}CCT^{r}T^{r}dT dT-3′ | SEQ ID NO: 41 |
| | 3′-dTdTAGGAGT^{r}CGGAGAAGAGGAA^{MeO}-p-5′ | SEQ ID NO: 42 |
| LC13-Md14 | 5′-U^{MeO}CCUCAGCCUCUUCUCCUU^{MeO}dT dT-3′ | SEQ ID NO: 43 |
| | 3′-dTdTAGGAGUCGGAGAAGAGGAAp-5′ | SEQ ID NO: 44 |
| LC13-Md15 | 5′-U^{MeO}C^{MeO}CUCAGCCUCUUCUCCU^{MeO}U^{MeO}dT dT-3′ | SEQ ID NO: 45 |
| | 3′-dTdTAGGAGUCGGAGAAGAGGAAp-5′ | SEQ ID NO: 46 |
| LC13-Md16 | 5′-U^{MeO}CCUCAGCCUCUUCUCCUUdT dT-3′ | SEQ ID NO: 47 |
| | 3′-dTdTAGGAGUCGGAGAAGAGGAA^{MeO}p-5′ | SEQ ID NO: 48 |

TABLE 2

| | | |
|---|---|---|
| siRNA | Nucleotide Sequence | Sequence ID |

| | | |
|---|---|---|
| LC20-WT | 5′-GGGUCGGAACCCAAGCUUA dTdT-3′ | SEQ ID NO: 49 |
| Unmodified | 3′-dAdT CCCAGCCUUGGGUUCGAAU-p-5′ | SEQ ID NO: 50 |
| LC20-19mer | 5′-GGGUCGGAACCCAAGCUUA-3′ | SEQ ID NO: 51 |
| No 3′ | 3′-CCCAGCCUUGGGUUCGAAU-p-5′ | SEQ ID NO: 52 |
| Overhangs | | |
| and | | |
| Unmodified | | |
| LC20- | 5′-G^{MeO}G^{MeO}GU^{MeO}C^{MeO}GGAAC^{MeO}C^{MeO}C^{MeO}AAGC^{MeO}U^{MeO}U^{MeO}A-3′ | SEQ ID NO: 53 |
| siSTABLE | 3′-UsUsC^{F}C^{F}C^{F}AGC^{F}C^{F}U^{F}U^{F}GGGU^{F}U^{F}C^{F}GAAU^{F}p-5′ | SEQ ID NO: 54 |
| LC20-MD3 | 5′-GGGUCGGAACCCAAGCUU^{MeO}A^{MeO} dTdT-3′ | SEQ ID NO: 55 |
| | 3′-dAdT C^{MeO}C^{MeO}CAGCCUUGGGUUCGAAU-p-5′ | SEQ ID NO: 56 |
| LC20-MD5 | 5′-G^{MeO}G^{MeO}GUCGGAACCCAAGCUUA dTdT-3′ | SEQ ID NO: 57 |
| | 3′dAdT CCCAGCCUUGGGUUCGAA^{MeO}U^{MeO}-p-5′ | SEQ ID NO: 58 |
| LC20-MD6 | 5′-GGGT^{r}CGGAACCCAAGCT^{r} U^{MeO} A^{MeO} dTdT-3′ | SEQ ID NO: 59 |
| | 3′-dAdT C^{MeO}C^{MeO}CAGCCT^{r}T^{r}GGGT^{r}T^{r}CGAAT^{r}-p-5′ | SEQ ID NO: 60 |
| LC20-MD8 | 5′-G^{MeO}G^{MeO}GT^{r}CGGAACCCAAGCT^{r}T^{r}A dTdT-3′ | SEQ ID NO: 61 |
| | 3′-dAdT CCCAGCCT^{r}T^{r}GGGT^{r}T^{r}CGAA^{MeO}U^{MeO}-p-5′ | SEQ ID NO: 62 |
| LC20-MD15 | 5′-G^{MeO}G^{MeO}GUCGGAACCCAAGCUUA dTdT-3′ | SEQ ID NO: 63 |
| | 3′-dAdT CCCAGCCUUGGGUUCGAAU-p-5′ | SEQ ID NO: 64 |
| LC20-MD17 | 5′-GGGUCGGAACCCAAGCUU A dTdT-3′ | SEQ ID NO: 65 |
| | 3′-dAdT C^{MeO}C^{MeO} CAGCCUUGGGUUCGAAAA^{MeO}U^{MeO}-p-5′ | SEQ ID NO: 66 |
| LC20-MD18 | 5′-G^{MeO}G^{MeO}GT^{r}CGGAACCCAAGCT^{r}U^{MeO}A^{MeO}dTdT-3′ | SEQ ID NO: 67 |
| | 3′-dAdT CCCAGCCT^{r}T^{r}GGGT^{r}T^{r}CGAT^{r}-p-5′ | SEQ ID NO: 68 |
| LC20-MD19 | 5′-GGGT^{r}CGGAACCCAAGCT^{r}T^{r}A dTdT-3′ | SEQ ID NO: 69 |
| | 3′-dAdT C^{MeO}C^{MeO} CAGCCT^{r}TrGGGT^{r}T^{r}CGAA^{MeO}U^{MeO}-p-5′ | SEQ ID NO: 70 |
| LC20-MD20 | 5′-GGGUCGGAACCCAAGCUU^{MeO}A^{MeO}dTdT-3′ | SEQ ID NO: 71 |
| | 3′-dAdTCCCAGCCUUGGGUUCGAAU-p-5′ | SEQ ID NO: 72 |
| LC20-MD21 | 5′-G^{MeO}G^{MeO}GT^{r}CGGAACCCAAGCT^{r}U^{MeO}A^{MeO}dTdT-3′ | SEQ ID NO: 73 |
| | 3′-CCCAGCCUUGGGUUCGAAU-p-5′ | SEQ ID NO: 74 |
| LC20-MD23 | 5′-GGGT^{r}CGGAACCCAAGCT^{r} U^{MeO}A^{MeO}dTdT-3′ | SEQ ID NO: 75 |
| | 3′-CCCAGCCUUGGGUUCGAAU-p-5′ | SEQ ID NO: 76 |

### EXAMPLE 5

### 2′-O-methyl Modified Ribonucleotides Improved siRNA Stability in Plasma

The purpose of this experiment was to determine whether 2′-O-methyl modified ribonucleotides and the substitution of uridines with ribothymidines (5-methyluridine) provide for greater siRNA stability against rat plasma ribonucleases. Improved stability was observed for both LC20 and LC13 indicating that these modifications will promote stability among all siRNAs universally. The siRNA duplexes listed in Table 1 and Table 2 in Example 4 were tested. The tables below show the stability rankings for the unmodified and modified forms of LC20 siRNA (Table 3) and LC13 siRNA (table 4) whereby a stability ranking of 1 is most stable.

TABLE 3

| | Stability |
|---|---|
| siRNA | Ranking |
| LC20-MD15 | 1 |
| LC20-MD5 | |
| LC20-MD3 | |
| LC20-MD6 | |
| LC20-MD19 | |
| LC20-MD8 | |
| LC20-MD17 | 2 |
| LC20-MD18 | |
| LC20-MD20 | |
| LC20-MD21 | 3 |
| LC20-MD23 | |
| LC20-MD22 | 4 |
| LC20-WT | |
| Unmodified | |
| LC20-19mer | |
| No 3′ overhangs | |
| and Unmodified | |

TABLE 4

| | Stability |
|---|---|
| siRNA | Ranking |
| LC13-Md4 | 1 |
| LC13-Md8 | |
| LC13-Md15 | |
| LC13-Md3 | |
| LC13-Md6 | 2 |
| LC13-Md7 | |
| LC13-Md5 | |
| LC13-Md14 | 3 |
| LC13-Md12 | |
| LC13-Md13 | 4 |
| LC13-Md16 | |
| LC13-19mer | |
| No 3′ overhangs | |
| and Unmodified | |
| LC20-WT | |
| Unmodified | |

A 20 μg aliquot of each siRNA duplex of Example 4 was mixed with 200 μl of fresh rat plasma incubated at 37° C. At various time points (0, 30, 60 and 20 min), 50 μl of the mixture was taken out and immediately extracted by phenol:chloroform. siRNAs were dried following precipitation by adding 2.5 volume of isopropanol alcohol and subsequent washing step with 70% ethanol. After resuspending in water and gel loading buffer the samples were analyzed gel electrophoresis on a 20% polyacrylamide gel, containing 7 M urea and subsequently visualized by ethidium bromide staining and quantified by densitometry.

Unmodified siRNA molecules were shown to be unstable in plasma. Surprisingly, however, siRNAs containing 2′ O-methyl modified ribonucleotides showed improved stability. More specifically, the greatest overall increase in LC20 siRNA stability was observed where two 2′O-methyl ribonucleotides were placed at the 5′-end and at the 3′-end, prior to the 3′ overhang, of the sense strand (LC20-MD15). Of note, the same modification to LC20 siRNA but in the anti-sense strand does not give the same degree of stability indicating that the stability enhancing effect of 2′ O-methyl modified ribonucleotides may be strand specific. The greatest overall increase in LC13 siRNA stability was observed in the presence of two 2′-O-methyl ribonucleotides at the 5′-end and at the 3′-end, prior to the 3′ overhand, of both the sense and anti-sense strands (LC13-Md4). Thus, siRNA duplex stability improves with the increasing presence of 2′O-methyl modified ribonucleotides at or near the ends of the siRNA duplex. In general, these data show the surprisingly and unexpected discovery that siRNA duplex stability in plasma is improved in the presence of 2′ O-methyl modified ribonucleotides at or near the ends of the siRNA duplex.

In general, the replacement of uridines with ribothymidines in combination with 2′ O-methyl modified ribonucleotides did not significantly affect siRNA duplex stability.

### EXAMPLE 6

### Ribothymidines Improve siRNA Stability by Increasing the Melting Temperature (T_{M}) of the siRNA Duplex

The purpose of this experiment was to determine the effect of incorporating ribothymidines in a double stranded RNA molecule in place of uridines on the melting temperature (T_{M}) of the siRNA molecule. A higher Tₘ correlates with increased siRNA duplex stability.

To determine the effect of replacing uridines with ribothymidines in the siRNA duplex upon melting temperature, thermal melting profiles were generated for four β-galactosidase (β-gal) siRNA molecules (table 5). These four β-gal siRNA duplexes differ only by the presence or absence of ribothymidines in the siRNA duplex. Where ᵣT is 5-methyluridine.

TABLE 5

| | | | |
|---|---|---|---|
| Duplex | Sequence | ID | Sequence ID |

| | | | |
|---|---|---|---|
| βgal-U | C U A C A C A A A U C | I | SEQ ID NO: 77 |
| (Homoduplex; | A G C G A U U U TT TT | | SEQ ID NO: 78 |
| WT) | G A U G U G U U U A G | | |
| | U C G C U A A A | | |
| βgal-T^{r} | C T^{r} A C A C A A A T^{r} | II | SEQ ID NO: 79 |
| (Homoduplex) | C A G C G A T^{r} T^{r} | | SEQ ID NO: 80 |
| | T^{r}TT TT G AT^{r} GT^{r} | | |
| | GT^{r}T^{r}T^{r} A GT^{r} C G C | | |
| | T^{r} A A A | | |
| βgal-U/ | C U A C A C A A A U C | III | SEQ ID NO: 81 |
| βgal-T^{r} | A G C G A U U U TT TT | | SEQ ID NO: 82 |
| | G AT^{r} GT^{r} GT^{r}T^{r} T^{r} A | | |
| | GT^{r} C G C T^{r} A A A | | |
| βgal-T^{r}/ | C T^{r} A C A C A A AT^{r} C | IV | SEQ ID NO: 83 |
| βgal-U | A G C G A T^{r}T^{r}T^{r} T T | | SEQ ID NO: 84 |
| | TT G A U G U G U U U | | |
| | A G U C G C U A A A | | |

The stock solutions of single stranded oligonucleotides were prepared by dissolving the selected sequences in 400 μL 10 mM buffer phosphate pH 7.0 containing 0.1 M NaCl and 0.1 mM EDTA and diluted (1 μL to 200 μL) with water and absorbencies (A₂₆₀) were measured and the contents were calculated. Also the integrity of the oligonucleotides was confirmed by HPLC analysis. To prepare the siRNA duplexes, the single stranded oligonucleotides were mixed and allowed to anneal. The UV absorption (A₂₆₀) for each siRNA duplex was measured and their values are as follows: I (0.28), II (0.54), III (0.31), and IV (0.45). To test for reproducibility, the melting profile study was done in duplicate.

The thermal melting profiles of the duplexes I, II, III and IV were recorded on Shimadzu UV-VIS 1601 with thermoelectrically temperature controlled through the Peltier device. The temperature was changed at the rate of 0.5° C./minute from 90° C. to 25° C. while the absorption recorded at 260 nm. The reverse experiment is also repeated. The “melting” process is a physical phenomenon. Therefore, the generated profile (90° to 25°) ought to be superimposed on the reverse (20° to 90°).

Differential curves were used to determine the melting point (Tₘ) of the duplexes. The shape of the curve defined by the derivative of α curve (versus 1/T) was used to make a robust determination of Tₘ and other thermodynamic data. Shimadzu TMSPC-8 and its associated software performed the needed calculations. The Tₘs have been listed in table 6. The duplicate experiment (2^{nd} Experiment; table not shown) had a similar thermal melting profile.

TABLE 6

| Duplex (ID) | Up/Down profile | Tₘ (° C.) |
|---|---|---|
| I (WT) | Up | 64.3 |
| I (WT) | Down | 65.6 |
| II | Up | 71.9 |
| II | Down | 72.8 |
| III | Up | 71.9 |
| III | Down | 72.9 |
| IV | Up | 67.8 |
| IV | Down | 67.8 |

As shown in table 7, a direct comparison was done between the duplicate experiments. The differences between the Tₘs derived from the 1^{st} Experiment and the 2^{nd} Experiment are shown in the far right column (ΔTₘ). Minimal to no difference was observed between the two experiments.

TABLE 7

| | Avg. Tₘ from 1^{st} Exp. | Avg. Tₘ from 2^{nd} Exp. | |
|---|---|---|---|
| Duplex | (° C.) | (° C.) | ΔTₘ |
| I (WT) | 64.9 | 64.9 | 0.0 |
| II | 72.35 | 72.25 | 0.1 |
| III | 72.4 | 69.8 | 2.6 |
| IV | 67.8 | 67.8 | 0.0 |

In conclusion, the incorporation of rT in the double stranded RNA in place of uridine residues increases the stability of the duplex by ˜0.6° C./rT incorporation. As shown in Table 7, duplex III (rT incorporated in the anti-sense strand only; a total of 7 rTs) melts at a temperature of approximately 4.9° higher than the wild type (duplex I). Duplex IV (rT incorporated in the sense stand only; a total of 5 rTs) melts at a temperature of 67.8° C., or 2.9° C. higher than the wild type. Finally, duplex II (rT incorporate in both the sense and anti-sense strands; 12 rTs) melts at about 72.3° C., or 7.4° C. higher than the wild type.

Thus, these data indicate the surprisingly and unexpected discovery that the Tₘ of the wild type β-gal RNA duplex is increased when the uridines are replaced with ribothymidines. Consequently, because of the increased Tₘ, the stability of the RNA duplex is increased.

### EXAMPLE 7

### siRNA Gene Knockdown Activity is Enhanced with 2′-O-methyl Ribonucleotides and Ribothymidines

The purpose of this experiment was to determine whether 2′O-methyl modified ribonucleotides in combination with the substitution of uridines with ribothymidines in the siRNA duplex would enhance its ability to downregulate target gene expression. siRNA knockdown activity was determined with a similar protocol as described in Example 2 except that siRNAs were transfected with the polynucleotide delivery-enhancing polypeptide PN73. PN73 was mixed with each siRNA at a 1:5 ratio. Each siRNA was tested at a concentration of 0.16 nM, 0.8 nM and 4 nM.

Unmodified forms of LC20 and LC13 with 3′ overhangs (LC20-WT and LC13-WT) were used as a baseline to determine whether modified siRNAs had increased target gene knockdown activity compared to unmodified siRNAs. Also, a random siRNA sequence was used as a negative control (Qneg).

FIG. 2 shows the knockdown activities for LC20-MD3, MD-6, MD-8, MD-15, MD-17, MD-18 and MD19. As shown in FIG. 2, the negative control, Qneg, showed no measurable knockdown activity. The greatest overall knockdown activity for LC20 was observed when two 2′O-methyl modified ribonucleotides were placed at the 5′-end of both the sense and anti-sense strands and all remaining uridines were converted to ribothymidines (e.g., LC20-MD6 and LC20-MD8).

Knockdown activities for modified LC13 siRNAs were also measured. The greatest overall knockdown activity for LC13 was observed with LC13-Md13 and LC13-Md15. LC13-Md13 has one 2′O-methyl modified ribonucleotide at the 5′-end of each strand and the remaining uridines are replaced with ribothymidines. LC13-Md15 has two 2′O-methyl modified ribonucleotides at the 5′-end and 3′-end of the sense strand.

In addition, the knockdown activity of siSTABLE and unmodified siRNAs were compared. The unmodified siRNAs provided a greater knockdown activity compared to the same siRNAs in siSTABLE form. Thus, siSTABLE modification of siRNAs does not provide increased knockdown activity over the unmodified form. Furthermore, siSTABLE siRNAs with 2′O-methyl modified ribonucleotides and/or ribothymidine substitutions did not change siSTABLE siRNA activity.

In general, these data show the surprisingly and unexpected discovery that siRNA duplex knockdown activity can be improved with the addition of 2′ O-methyl modified ribonucleotides at or near the ends of the siRNA duplex and where ribothymidines are substituted for uridines within the siRNA molecule.

### EXAMPLE 8

### siRNA Off Target Effect is Minimized with 2′-O-methyl Ribonucleotides and Ribothymidines

The purpose of this experiment was to determine whether siRNA gene target specificity could be enhanced with 2′-O-methyl modified ribonucleotides and ribothymidine substitutions in the siRNA duplex. Although siRNA is a powerful technique used to disrupt the expression of target genes, an undesired consequence of this method is that it may also effect the expression of non-target genes (off-target effect). Thus, to determine if the off-target effect of siRNA molecules could be minimized with the addition of 2′-O-methyl ribonucleotides and ribothymidines, an off-target profile was generated for 5 different siRNAs that target the human tumor necrosis factor-α (TNF-α) mRNA. The modified siRNA was based on the MD8 modification listed in tables 1 and 2 of Example 4. An example of an LC20-siSTABLEv2 modified siRNA is shown as table 2 of Example 4.

Agilent microarrays were used and consisted of 60-mer probe oligonucleotides (targets) representing ˜18,500 well-characterized, full-length human genes. The unmodified siRNA candidates showed an off-target effect of between 5 to 84 gene expression changes out of a total of 18,500 genes. An off-target gene effect was counted when a 2-fold change (up or down) in gene expression was observed. The siSTABLEv2 modified siRNAs showed a decreased off-target effect. Surprisingly, the siRNA candidates with the full ribothymidine substitution, 3′-ends with 2 base dT overhangs, and 5′-end dinucleotide 2′-O-methyl substituted riboses showed minimal off-target effects (table 8). In particular, TNFα-2, TNFα-17 and LC20 siRNAs with 2′O-methyl modified ribonucleotides and ribothymidines showed no off-target effect.

TABLE 8

| | Unmodified | | Modified siRNA |
|---|---|---|---|
| | siRNA Off- | siSTABLEv2 | Off-Target Effect |
| siRNA Canidate | Target Effect | Modified | (2′-O-methyl + riboT) |
| TNFα-2 | 33 | 2 | 0 |
| TNFα-9 | 69 | 3 | 4 |
| TNFα-17 | 84 | 2 | 0 |
| LC17 | 51 | 9 | 12 |
| LC20 | 5 | 3 | 0 |

The siRNA modification had a significant effect on reducing off-target responses. From this data, the extent of G:U base pairing in all the identified siRNA off-target interactions should be able to be evaluated and therefore, the potential ribothymidine substitutions needed to eliminate the off-target effects by the suppression of G:U wobble should be able to be ascertained.

These data show the surprisingly and unexpected discovery that the siRNA off target effect can be minimized or even ablated by the addition of 2′-O-methyl modified ribonucleotides and ribothymidine substitutions.

### EXAMPLE 9

### Interferon Response

Interferon responsiveness is a potential side-effect of tranfecting cells with siRNAs. Thus, a study was performed in vitro to assess whether various isoforms of the LC20 siRNA would elicit an interferon response. Both unmodified and modified of the 19-mer LC20 siRNA and 21-mer LC20 siRNA were tested. The 21-mer LC20 siRNA contains 2 base pair 5′-end overhangs while the 19-mer LC20 siRNA does not.

The unmodified 21-mer and 19-mer forms of the LC20 siRNA did not elicit an interferon response. Furthermore, the 21-mer LC20-MD8 modified siRNA, which includes two 2′O-methyl modified ribonucleotides at the 5′-end of each strand and the replacement of all remaining unmodified uridines with ribothymidines, did not elicit an interferon response. However, the identical modification of LC20 but in the 19-mer length induced an interferon response.

The teachings of all of references cited herein including patents, patent applications and journal articles are incorporated herein in their entirety by reference.

What is claimed is:

1. A method of minimizing off-target effects of siRNA, comprising preparing double-stranded RNA (dsRNA) having a sense strand that is homologous to a sequence of a target gene and an anti-sense strand that is complementary to said sense strand, and having at least one pyrimidine replaced by a 5′-methyl-pyrimidine, and contacting said dsRNA with a cell capable of expressing said target gene.

2. The method of claim 1, wherein the 5′-methyl-pyrimidine is ribothymidine.

3. The method of claim 2, wherein at least three of the uridines of the siRNA sequence are replaced by ribothymidines.

4. The method of claim 2, wherein at least three of the uridines of the sense strand of the siRNA sequence are replaced by ribothymidines.

5. The method of claim 2, wherein at least three of the uridines of the antisense strand of the siRNA sequence are replaced by ribothymidines.

6. The method of claim 2, wherein all of the uridines in the siRNA sequence are replaced by ribothymidines.

7. The method of claim 2, wherein the siRNA molecule has a 3′ overhang.

8. The method of claim 2, wherein the siRNA molecule is blunt ended.

9. A method of increasing stability of siRNA, comprising preparing double-stranded RNA (dsRNA) having a sense strand that is homologous to a sequence of a target gene and an anti-sense strand that is complementary to said sense strand, and having at least one pyrimidine replaced by a 5′-methyl-pyrimidine, and contacting said dsRNA with a biological sample.

10. The method of claim 9, wherein the biological sample is blood serum or plasma.

11. The method of claim 9, wherein at least three of the uridines of the sense strand of the siRNA sequence are replaced by ribothymidines.

12. The method of claim 9, wherein at least three of the uridines of the antisense strand of the siRNA sequence are replaced by ribothymidines.

13. The method of claim 9, wherein at least three of the uridines in the siRNA sequence are replaced by ribothymidines.
